(19) United States
(12) Patent Application Publication
(10) Pub. No.: US 2006/0121083 A1
(43) Pub. Date: Jun. 8, 2006

(54) Anti-microbial medical implants and uses thereof

(75) Inventor: Amram Mor, Nesher (IL)

Correspondence Address: Martin Moynihan;Prtsi Inc, PO Box 16446, Arlington, VA, 22215, US

(73) Assignee: Technion Research & Development Foundation Ltd., Haifa (IL)

(21) Appl. No.: 10/564,619

(22) PCT Filed: Jul. 15, 2004
(86) PCT No.: PCT/IL04/00642

§ 371 Date: Jan. 13, 2006

Related U.S. Application Data
(60) Provisional application No. 60/487,956, filed on Jul. 18, 2003.

(51) Int. Cl. A61K 31/497; A61F 2/00; A61K 31/496; A61K 38/17

(52) U.S. Cl. 424/426; 514/12; 514/252.13

(57) ABSTRACT

A medical device or implant is disclosed. The medical device or implant comprises a body having at least one surface coated with, or including a peptide having at least 9 amino acid residues and less than 51 amino acid residues, the peptide including an amino acid sequence derived from an antimicrobial peptide.

### FIELD AND BACKGROUND OF THE INVENTION

The present invention relates to medical devices/implants which include/are coated with anti-microbial compounds, to methods of fabricating such medical implants/devices, and to methods of using such medical implants/devices to prevent microbial infection in a subject in need of implantation of a medical implant. More particularly, embodiments of the present invention relate to synthetic carbon polymer grafts coated with antibacterial peptides, to methods of fabricating such grafts, and to methods of using such grafts to prevent staphylococcal infection in subjects receiving medical implants.

Infection of medical implants/devices, specifically prosthetic vascular grafts, as well as venous or urinary catheters, prosthetic heart valves, orthopaedic devices, and contact lenses, is an ever-growing problem and concern. For example, prosthetic vascular grafts are a source of significant clinical morbidity and mortality upon infection [Bradley S F., 2002. Clin Infect Dis. 34, 211; Lowy, F. D. 1998. N. Engl. J. Med. 339: 520-532; Huebner, J., Goldman, D. A. 1999. Ann. Rev. Med. 50: 233-236 Goldstone, J. and W. S. Moore, Am. J. Surg. 128: 225 (1974); Liekweg et al., Surgery 81: 335 (1977); Bunt, T. J., Surgery 93: 733 (1983); Golan, J. F., Infect. Dis. Clin. N. Am., 3: 247 (1989); Sugarman, B. and E. J. Young, Infect. Dis. Clin. N. Am. 3: 187 (1989)]. Graft infection is associated with significant lethality and morbidity with graft infection occuring in 2-6% of all clean cases performed [Hoffert et al., Arch. Surg. 90: 427 (1965); Fry, W. L. and S. M. Lindenauer, Arch. Surg. 94: 600 (1966); Rittenhouse et al. Ann. Surg. 170: 87 (1969); Drapanas et al., Ann. Surg. 172: 351 (1970); Szilagyi et al., Ann. Surg. 176: 321 (1972)].

Infectious inoculation of medical implants/devices presumably occurs at the time of implantation or as a result of transient bacteremia in the immediate post-operative period [Cheri et al., J. Vasc. Surg. 14: 521 (1991)]. Infections associated with medical implants are often associated with formation of bacterial biofilms on surfaces of medical devices (Costerton, J. W. et al., 1999. Science 284: 1318-1322; Marr, K. A. 2000. Seminars in Dialysis. 13: 23-29; Schierholz, J. M., Beuth, J. 2001. J. Hosp. Infect. 49: 87-93; Linnola, R. 2001. Ophthalmology 108: 1518-1519). Peri-operative parental antibiotics, while having a defined role in wound infection prophylaxis, often fail to permeate the avascular spaces immediately around prosthetic grafts as well as the carbohydrate-rich bacterial biofilm once pathogens have adhered [Gristina, A. G., Science 237: 1585 (1987); Kaiser et al., Ann. Surg. 188: 283 (1978); Greco, R. S., J. Vasc. Surg. 13: 5 (1991); Bandyk et al., J. Vasc. Surg. 13: 575 (1991)].

The two main types of bacteria responsible for graft infection are the coagulase negative, common skin-inhabitant bacteria Staphylococcus aureus (S. aureus) and Staphylococcus epidermidis (S. epidermidis) (Costerton, J. W. et al., 1999. Science 284: 1318-1322; Marr, K. A. 2000. Seminars in Dialysis. 13: 23-29; Bradley S F., 2002. Clin Infect Dis. 34, 211; Lowy, F. D. 1998. N. Engl. J. Med. 339: 520-532; Huebner, J., Goldman, D. A. 1999. Ann. Rev. Med. 50: 233-236). S. aureus has been shown to be responsible for 65-100% of acute (days to weeks) infections. Typically, these infections develop rapidly and generate an intense response by the host defense mechanisms. An ever-increasing problem which has been documented both in animal models and in humans is the susceptibility of vascular prostheses to later (months to years) infection. S. epidermidis has emerged as the leading isolate from infection vascular conduits (20 to 60 percent) with infection appearing late after implantation. Both of these instances are clearly not affected by low level antibiotic transiently occurring at the time of surgery. The late-appearing vascular graft infections are thus one of the most feared complications following surgical implantation of vascular grafts, frequently resulting in prolonged hospitalization, organ failure, amputation, and death (Barie, P. S. 1998. World J Surg. 22: 118-126; Henke, P. K. et al., 1998. Am Surg 64: 39-45).

There is therefore, an urgent need for novel and optimal means of controlling infections, such as staphylococcal infection, resulting from implantation of medical implants/devices such as vascular grafts.

Effective strategies for the prevention of infection associated with medical implants/devices remain suboptimal, and vary from device to device, and are complicated by the capacity of staphylococci to produce a biofilm rendering them resistant to conventional antimicrobial agents (Stewart, P. S. and Costerton, J. W. 2001. Lancet. 358: 135-138; Mah, T. F., O'Toole, G. A. 2001. Trends Microbiol. 9: 34-39). The principal approach currently employed for treating nosocomial infections associated with medical implants/devices has been prophylactic/systemic administration of antibiotics. Recent prophylactic strategies have suggested the use of antimicrobial agents bound at high concentrations to prosthetic grafts to supplement systemic administration of antimicrobial agents (Sardelic, F. et al., 1996. Cardiovasc. Surg. 4: 389-392; Carratala, J. 2002. Clin. Microbiol. Infect. 8: 282-9; Tiller, J C. et al., 2001. Proc Natl Acad Sci USA. 98: 5981-5). Several studies have focused on developing new prosthetic materials capable of reducing adhesion or survival of bacteria thereupon (Tiller, J C. et al., 2001. Proc Natl Acad Sci USA. 98: 5981-5; Gottenbos, B et al., 2001. J Antimicrob Chemother. 48: 7-13).

Resistance of bacterial biofilms to antimicrobial agents is based on a multi-cellular mechanism that relies on exchange of chemical signals between cells in a process known as quorum sensing. Thus, one potentially potent approach which has been proposed for treating bacterial infections associated with biofilm formation involves the development of agents capable of interfering with bacterial cell-cell communication (Miller, M. B., Bassler, B. L. 2001. Annu Rev Microbiol. 55:165-199). Recently, a seven amino acid peptide termed RNAIII-inhibiting peptide (RIP) having the capacity to treat diseases caused by S. aureus and S. epidermidis was described (Balaban, N. et al., 1998. Science. 280: 438-440; Balaban, N. et al., 2000. Peptides. 21: 1301-1311; Gov, Y. et al.,2001. Peptides. 22: 1609-20; Vieira-da-Motta, O. et al., 2001. Peptides. 22: 1621-1627; Balaban, N. et al., 2003. J Infect Dis. 187: 625-30; Giacometti, A. et al., 2003. Antimicrobial Agents and Chemotherapy, In Press; Balaban, N. et al., 2003. Kidney Int. 63: 340-345). RIP was shown to inhibit S. aureus and S. epidermidis pathogenic biofilm formation (Balaban, N. et al., 2003. Kidney Int. 63: 340-345) and toxin production (Vieira-da-Motta, O. et al., 2001. Peptides. 22: 1621-1627) by disrupting quorum sensing mechanisms through inhibition of phosphorylation of target of RNAIII-activating protein (TRAP; Balaban, N. et al., 2001. J Biol Chem. 276: 2658-2667). RIP was shown to be effective against all staphylococcal strain tested so far (Balaban, N. et al., 2000. Peptides. 21: 1301-1311; Gov, Y. et al., 2001. Peptides. 22: 1609-20; Vieira-da-Motta, O. et al., 2001. Peptides. 22: 1621-162; Balaban, N. et al., 2003. J Infect Dis. 187: 625-30; Giacometti, A. et al., 2003. Antimicrobial Agents and Chemotherapy, In Press), presumably by virtue of TRAP being highly conserved among, as well as being protein unique to, staphylococci. RIP does not directly kill the bacteria but interferes with its signal transduction, thus making it non pathogenic.

Cationic antimicrobial peptides, which are ubiquitously produced in nature (Boman, H. G. 1995. Annu. Rev. Immunol. 13: 61-92; Nicolas, P. and Mor, A. 1995. Annu. Rev. Immunol. 49: 277-304; Hancock, R. E. 1997. The Lancet. 349: 418-22; Hancock, R. E. and Lehrer, R. 1998. Trends Biotechnol. 16: 82-90; Ganz, T. and Lehrer, R. 1998. Curr. Opin. Immunol. 10: 41-44; Andreu, D., Rivas, L. 1998. Biopolymers. 47: 415-33; Levy, O. 2000. Blood. 96: 2664-72; Tossi, A. et al., 2000. Biopolymers. 55: 4-30), play important roles in innate immunity, and, as such, their use has been proposed in various anti-bacterial applications (Mor, A. 2001. The Kirk-Othmer Encyclopedia of Chemical Technology. John Wiley & Sons. http:www3.interscience.wiley.com:8095/articles/peptwise.aol/frame.html; Zasloff, M. 2002. Nature. 415: 389-95). Antimicrobial peptides are thought to exert their action via targeting and disruption of the cytoplasmic membrane. By virtue of their being cationic, they are able to interact electrostatically with the negatively charged phospholipid headgroups, and to concomitantly insert into the membrane bilayer so as to lead to its disruption (Ludtke, S J. et al., 1994. Biochim. Biophys. Acta. 1190: 181-4; Heller, W. T. et al., 1998. Biochemistry. 37: 17331-38; Huang, H. W. 2000. Biochemistry. 39: 8347-52; Shai, Y. 2002. Biopolymers. 66: 236-48). Although the steps involved in this mechanism remain to be delineated, there is a large body of experimental data demonstrating that there is a direct correlation between the antibiotic effect of these peptides and their capacity to increase plasma membrane permeability, and concomitant conductance of ions across lipid bilayers and dissipation the trans-membrane electric potential (Pouny, Y. et al., 1992. J. Biol. Chem. 31: 12416-23; Levy, O. 2000. Blood. 96: 2664-72; Moll, G. N. et al., 2000. Biochemistry. 39: 11907-12; Oren, Z. and Shai, Y. 2000. Biochemistry. 39: 6103-6114; Friedrich, C. L. et al., 2000. Antimicrob. Agents Chemother. 44: 2086-92; Sokolov, Y. et al., 1999. Biochim. Biophys. Acta. 1420: 23-9; Oren, Z. and Shai, Y. 1998. Biopolymers. 47: 451-63; Shai, Y. 1999. Biochim. Biophys. Acta. 1462: 55-70; Levy, O. 2000. Blood. 96: 2664-72; Gazit, E. et al., 1995. Biochemistry. 34: 11479-88; Duclohier, H. and Wroblewski, H. 2001. J. Membr. Biol. 184: 1-12). Thus, while the precise mechanism of action of such peptides is not fully understood, their microbicidal effect is believed to result from their capacity to disrupt the ordered membrane structure of target cells. Antimicrobial peptides display preferential targeting to bacterial cells as opposed to mammalian cells via a still ill-defined mechanism which is believed to involve exploitation of the differences in the properties of membranes of target versus non-target cells, such as membrane fluidity and negative charge density (Andreu, D., Rivas, L. 1998. Biopolymers. 47: 415-33; Zasloff, M. 2002. Nature. 415: 389-95; Maloy, L. W. and Kari U. P. 1995. Biopolymers. 37: 105-22; Chen, J. et al., 2000. Biopolymers. 55: 88-98). Numerous studies have demonstrated that the peptides' physicochemical properties, i.e., amphipathy, positive charge content and hydrophobicity are the main factors affecting membrane-lysis activity (Andreu, D., Rivas, L. 1998. Biopolymers. 47: 415-33; Maloy, L. W. and Kari U. P. 1995. Biopolymers. 37: 105-22; Chen, J. et al., 2000. Biopolymers. 55: 88-98; Blondelle, E. S. and Lohner, K. 2000. Biopolymers. 55: 74-87). Accordingly, isomers composed of all D-amino acids are as active as the L-enantiomers, implying that the mechanism of action is not mediated by interaction with a stereo-specific receptor. These properties therefore enable cationic antimicrobial peptides to escape microbial mechanisms involved in antibiotic resistance or multidrug resistance (Chen, J. et al., 2000. Biopolymers. 55: 88-98; Ge, Y. et al., 1999. Antimicrob. Agents Chemother. 43: 782-788; Navon-Venezia, S. et al., 2002. Antimicrob. Agents Chemother. 46: 689-694).

Dermaseptins are a large family of linear polycationic antibacterial peptides from frog skin (Mor, A. et al., 1991. Biochemistry. 30: 8824-30; Mor, A. and Nicolas, P. 1994. Eur. J. Biochem. 219: 145-54; Mor, A. et al., 1994. Biochemistry. 33: 6642-50; Brand, G. D. et al., 2002. J Biol Chem. 277: 4933240) having potent cytolytic activity which is believed to result from interaction of their N-terminal domain with the plasma membranes of target cells (Mor, A. et al., 1994. J. Biol. Chem. 269: 31635-40; Mor, A. and Nicolas, P. 1994. J. Biol. Chem. 269: 1934-39). Recent investigations have investigated the relationship between physical properties, such as structure and organization in solution, of dermaseptin S4 and its interaction with target membranes (Feder, R. et al., 2000. J. Biol. Chem. 275: 4230-38; Kustanovich, I. et al., 2002. J. Biol. Chem. 277: 16941-51). Thus, in view of the biophysical properties thereof, peptides derived from dermaseptin S4, have therefore been proposed to have potential utility in the fabrication of medical implants/devices being optimally capable of preventing infection with microbial pathogens. Dermaseptin S4 derivatives have been designed that maintain the amphipathic alpha-helical structure of the parent peptide, bind avidly to model membranes with association affinity constants (K_{A}) in the range of 10⁵ to 10⁷ M⁻¹ and exert cytolytic activity against a variety of pathogens in-vitro (Feder, R. et al, 2001. Peptides. 22: 1683-90; Navon-Venezia, S. et al., 2002. Antimicrob. Agents Chemother. 46: 689-694; PCT publication WO 01/10887 to the present inventors). The efficacy of DD13 and other dermaseptin derivatives as an antibiotic was demonstrated in-vivo in a mouse model of P. aeruginosa intraperitoneal infection (Navon-Venezia, S. et al., 2002. Antimicrob. Agents Chemother. 46: 689-694).

As described above, cationic antimicrobial peptides are superior to antibiotics by virtue of their potent antimicrobial activity and their biophysical mode of action involving targeting cell membrane lipids which enables them to be unaffected or minimally affected by microbial defense mechanisms involving multi-drug resistance and/or mutational adaptation. Thus, a potentially optimal strategy for preventing microbial infection resulting from implantation of medical implants/devices would be via incorporation or coating of antimicrobial peptides.

Several approaches involving the use of antimicrobial peptides to prevent microbial infection of medical implants/devices have been described in the prior art.

In one approach, coating of Dacron vascular grafts with the antimicrobial peptide temporin A, alone or in combination with RIP, has been attempted to prevent staphylococcal infection of the grafts post-implantation in-vivo (Cirioni O. et al., 2003. Circulation 108:767-71).

In another approach, coating of Dacron grafts with ranalexin or buforin II alone or with perioperative intraperitoneal cefazolin prophylaxis has been attempted as prophylaxis against methicillin-susceptible or methicillin-resistant S. epidermidis vascular graft infection. (Giacometti A. et al, 2000. Antimicrob Agents Chemother. 44:3306-9).

In a further approach, coating of Dacron grafts with the antimicrobial peptide nisin, alone or in combination with RIP, has been attempted to prevent infection of such grafts with S. epidermidis ATCC 12228 or a clinical isolate of methicillin-resistant S. epidermidis following in-vivo implantation (Ghiselli R. et al., 2004. Eur J Vasc Endovasc Surg. 27:603-7).

All of the prior art approaches, however suffer from various critical limitations. In particular, no prior art approach has achieved optimal prevention of in-vivo infection of medical implants/devices by methicillin-resistant S. aureus or methicillin-resistant S. epidermidis, the most feared type of complication following implantation of various types of medical implants/devices, including vascular grafts.

Thus, all prior art approaches have failed to provide an adequate solution for using antimicrobial peptides to optimally prevent infection of medical implants/devices by microbial pathogens.

There is thus a widely recognized need for, and it would be highly advantageous to have an optimal method of using antimicrobial peptides for preventing infection of medical implants/devices, devoid of the above limitation.

### SUMMARY OF THE INVENTION

According to one aspect of the present invention there is provided a method of fabricating a medical device or implant capable of killing, or preventing a growth of, a microbial pathogen, the method comprising contacting at least one surface of a body of the medical device or implant with a peptide having at least 9 amino acid residues and less than 51 amino acid residues, the peptide including an amino acid sequence selected from the group consisting of SEQ ID NOs: 1-5, thereby rendering the surface of the medical implant capable of killing, or preventing the growth of, the microbial pathogen.

According to further features in preferred embodiments of the invention described below, contacting the at least one surface of the medical device or implant with the peptide is effected by exposing the at least one surface of the medical device or implant with a solution of the peptide, wherein a concentration of the peptide in the solution is selected from a range of 1 to 500 micrograms per milliliter.

According to still further features in the described preferred embodiments, exposing the at least one surface of the medical device or implant with the solution of the peptide is effected for a duration selected from a range of 0.05 to 50 hours.

According to still further features in the described preferred embodiments, the solution further comprises an antibiotic.

According to still further features in the described preferred embodiments, the concentration of the antibiotic in the solution is selected from a range of 0.5 to 50 micrograms per milliliter.

According to another aspect of the present invention there is provided a method of preventing microbial infection in a subject in need of implantation of a medical implant, the method comprising administering to the subject a medical implant comprising a body having at least one surface, the at least one surface being coated with, or including a peptide having at least 9 amino acid residues and less than 51 amino acid residues, the peptide including an amino acid sequence selected from the group consisting of SEQ ID NOs: 1-5, thereby treating the subject in need thereof with the medical implant.

According to yet another aspect of the present invention there is provided a medical device or implant comprising a body having at least one surface coated with, or including a peptide having at least 9 amino acid residues and less than 51 amino acid residues, the peptide including an amino acid sequence selected from the group consisting of SEQ ID NOs: 1-5.

According to further features in preferred embodiments of the invention described below, the peptide is amidated.

According to still further features in the described preferred embodiments, the at least one surface is coated with the peptide at a surface density selected from a range of 0.4 to 275 micrograms per square centimeter.

According to still further features in the described preferred embodiments, the at least one surface is composed of a synthetic carbon polymer and/or a polypeptide.

According to still further features in the described preferred embodiments, the medical device or implant is a vascular graft.

According to still further features in the described preferred embodiments, the at least one surface is also coated with or also includes an antibiotic.

According to still further features in the described preferred embodiments, the antibiotic is rifampin.

The present invention successfully addresses the shortcomings of the presently known configurations by providing a medical device/implant having the capacity to optimally prevent infection by a microbial pathogen, by providing an optimal method of fabricating such a medical implant/device, and by providing an optimal method of preventing microbial infection in a subject in need of implantation of a medical implant.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic diagram depicting a general configuration of the medical implant device of the present invention (cylinder cross-section).

FIGS. 2a-b are data plots depicting that DD13 displays optimal dose-dependent in-vivo anti-infective activity relative to RIP against methicillin-resistant staphylococcal strains. Dacron™ grafts were pre-soaked in DD13 or RIP (FIGS. 2a-b, respectively), at the designated concentrations and implanted in rats. Grafts were pre-soaked in saline as a negative control. Following, presoaking, grafts were inoculated with methicillin-resistant S. aureus. (MRSA) or a clinical isolate of methicillin-resistant S. epidermidis (MRSE). Grafts were removed after a week and assessed for bacterial load. Plots show typical counts of viable CFUs for MRSA (triangles) or MRSE (circles).

FIG. 3. is a bar-graph depicting optimal synergistic prevention of growth of bacterial pathogens in-vivo by a combination of DD13 and rifampin, relative to RIP, DD13 or rifampin used singly. Grafts pre-soaked in saline (control), rifampin alone (5 mg/L) or rifampin combined with either RIP or DD13 (10 mg/L) were implanted in rats and inoculated with MRSA or MRSE. Grafts were removed after a week and assessed for bacterial load. Plots show the resulting counts of viable CFU. A star indicates negative quantitative cultures.

FIG. 4 is a bar-graph depicting the optimal capacity of DD13 to bind to Dacron™ grafts relative to RIP. Collagen-sealed Dacron™ grafts were soaked in a 50 mg/L solution of the indicated peptide. The quantity of bound peptide was estimated from the unbound fraction which was analyzed via reversed-phase HPLC. Peptide identification was based on retention time and spectral analysis. Unbound peptide quantity was determined after area integration of the UV absorbing peak (220 nm) and comparison with standard curves of known concentrations (Feder, R. et al., 2000. J. Biol. Chem. 275: 4230-38). Error bars indicate standard deviations of the mean determined from four independent experiments.

FIGS. 5a-b are data plots depicting the optimal anti-bacterial activity of DD13 against S. aureus cells in-vitro relative to RIP (FIG. 4a), and an analysis of the capacity of DD13 to inhibit expression of RNAIII (FIG. 4b). A culture of about 2×10⁷ S. aureus cells expressing an maiii::blaZ fusion construct was grown with peptides or control buffer, and divided into two portions. One portion was diluted in saline and streaked onto LB agar plates to determine CFU (FIG. 4a). The other portion was used to determine the effects of the peptides on RNAIII synthesis (FIG. 4b) by adding the beta-lactamase substrate nitrocefin, and measuring OD 490 nm/650 nm (Gov, Y. et al., 2001. Peptides. 22: 1609-20). Error bars indicate standard deviations from the mean as determined from two independent experiments performed in triplicate. The inset in FIG. 4b depicts percent inhibition of RNAIII expression at low peptide concentrations.

### DESCRIPTION OF THE PREFERRED EMBODIMENTS

The present invention is of medical implants/devices having a surface which includes or is coated with an antimicrobial peptide capable of optimally killing/preventing the growth of a microbial pathogen, of methods of fabricating such medical implants/devices, and of methods of using such medical implants/devices for preventing microbial infection in a subject in need of implantation of a medical implant.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Therapeutic implantation of medical implants/devices, such as synthetic vascular grafts, is associated with risk of highly debilitating or lethal infection with dangerous pathogens, such as methicillin-resistant Staphylococcus aureus (S. aureus) or Staphylococcus epidermidis (S. epidermidis).

Coating of such medical implants/devices with a variety of antimicrobial peptides, alone or in combination with non-antimicrobial peptide antibiotics, has been suggested or attempted, as a means for reducing the risk of such infection. Such coating has been attempted with antimicrobial peptides such as temporin A or ranalexin, alone or in combination with RIP; or buforin II or nisin.

However, all such prior art medical implants/devices suffer from various drawbacks, in particular failure to provide optimal protection against post-implantation infection by methicillin-resistant S. aureus or methicillin-resistant S. epidermidis, the most feared type of complication following implantation of medical implants/devices, such as vascular grafts.

While reducing the present invention to practice, the present inventors demonstrated for the first time that coating a collagen-coated medical implant/device with a dermaseptin S4-derived antimicrobial peptide, alone or optimally in combination with the antibiotic rifampin, will optimally reduce the risk that in-vivo implantation of the implant/device will be associated with infection by either methicillin-resistant S. aureus or methicillin-resistant S. epidermidis strains, which remains a potentially lethal complication associated with implantation of medical implants/devices, such as synthetic vascular grafts. As such, implantation of the medical implants/devices of the present invention is associated with minimally low risk of such a complication relative to implantation of prior art medical implants/devices.

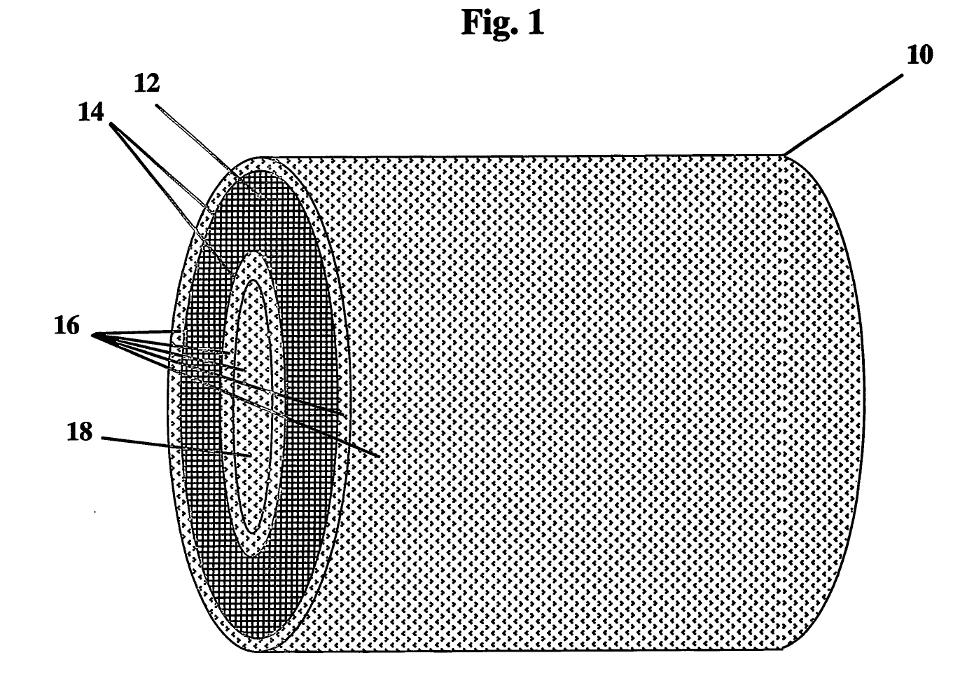

Thus according to one embodiment of the present invention and as specifically shown in FIG. 1, there is provided a medical device or implant which is referred to hereinunder as graft 10. Graft 10 comprises device body 12 having at least one surface 14 coated with, or including peptide 16. In the embodiment shown in FIG. 1, device body 12 is configured as a tubular element having lumen 18. Device body 12 may have any size and shape configuration, in accordance with the intended use, and nature and size of implantation site, and can be configured for long-term or transient implantation. Device body 12 may be designed and configured as any of various medical implants/devices, as described further hereinbelow. In the embodiment of the medical implant/device of the present invention depicted in FIG. 1, graft 10 is designed and configured as a cylindrical vascular graft for implantation in a vascular tissue region of a subject. Physical dimensions of device body 12 are selected according to the target tissue. Where graft 10 is a cylindrical vascular graft, lumen 18 is of an inner cross sectional area of about 7 to 700 square millimeters or any cross sectional area or diameter which is substantially equivalent to an inner cross sectional area or diameter of a blood vessel. For example aortic, esophageal, tracheal, and colonic stents may have dimensions of about 25 mm in width/diameter and lengths of about 100 mm or even longer.

As used herein, the phrase “medical implant/device” refers to any medical device or apparatus which, permanently or transiently, is implanted within, and/or which is contacted with, the body of a subject.

A peptide of the present invention which is “included” in surface 14 is integrated therein and/or is fabricated therewith.

As used herein, the term “subject” refers to a vertebrate, more preferably to a homeotherm, more preferably to a mammal and most preferably to a human.

As used herein, the phrase “tissue region” refers to any tissue of a subject. For example, the tissue region may be a vascular vessel/duct within a vascular/ductal system, such as a vascular/ductal network, the esophagus, the trachea, biliary ducts, the, urethra, ureters or the lymphatic system. The tissue region according to the present invention may be normal, ischemic, necrotic, neoplastic, hyperplastic, and the like.

Device body 12 may be composed of a variety of conventional materials. These include biocompatible metals such as polyethylene terephthalate fiber (commercially known as Dacron), stainless steel, tantalum, titanium, nitinol, gold, platinum, inconel, iridium, silver, tungsten, or alloys thereof; carbon or carbon fibers; cellulose acetate, cellulose nitrate, silicone, polyethylene, teraphthalate, polyurethane, polyamide, polyester, polyorthoester, polyanhydride, polyether sulfone, polycarbonate, polypropylene, high molecular weight polyethylene, polytetrafluoroethylene (PTFE), expanded polytetrafluoroethylene (ePTFE), or polyester fibers or another biocompatible polymeric material or mixtures or co-polymers of these; polylactic acid, polyglycolic acid or co-polymers thereof, a polyanhydride, polycaprolactone, polyhydroxybutyrate valerate or another biodegradable polymer or mixtures or co-polymers of these; a protein, an extra-cellular matrix component, collagen, fibrin, albumin or suitable mixtures thereof.

Alternately, device body 12 may be composed of processed blood vessels derived from an animal or a human.

Preferably device body 12 is substantially composed of a synthetic carbon polymer, most preferably Dacron.

In a preferred embodiment, device body 12 is configured as a textile, preferably a flexible woven or braided textile. Dacron-based textile vascular grafts are commonly employed in the art, as referred to in numerous references provided herein.

Surface 14 serves as a surface for attaching peptide 16. Preferably surface 14 is made of or is coated with a biocompatible material, which is non-immunogenic. Surface 14 can include a biodegradable material, such as a polypeptide. The biodegradable material used may be selected based upon its clearance rate and toxicity of degradation products. For example, high molecular weight biomaterials can be used when clot target sites are involved. High molecular weight hydrophilic polymers, triblock polymers, hyaluronic acid, and albumin demonstrate non-toxic post-degradation characteristics. The biodegradable material can be a lubricant, and/or a hydrophilic (albumin, triblock polymer, hyaluronic acid, heparin, PEOs, PEGs, polyurethanes, etc., or mixtures thereof), and/or natural (gelatin, fibrin, fibrinogen, collagen, fibronectin, etc., or mixtures thereof) or synthetic (silica-based) hydrophobic adhesive biomaterial, and/or a lipid-based biomaterial (phospholipids, lipid extracts, triglyceride films, polymers of fatty acids, waxes, sphingolipids, sterols, glycolipids, etc., or mixtures of thereof). Surface 14 may advantageously include metallic clusters or colloids, such as colloidal gold for attachment of peptide 16.

Surface 14 is preferably composed of a synthetic carbon polymer and/or a polypeptide, preferably both. Preferably, the synthetic carbon polymer is Dacron. Preferably, the polypeptide is albumin.

Preferably, surface 14 is also coated with or also includes an antibiotic, most preferably rifampin.

A graft 10 having an albumin-coated surface 14 is commercially available. A preferred example of such a Dacron-based graft is Albograft™ which may be commercially obtained from Sorin Biomedica Cardio, S.p.A., Saluggia VC, Italy.

As is mentioned hereinabove, surface 14 is coated with or includes peptide 16 which serves to kill or prevent the growth of microbial pathogens, such as S. aureus and S. epidermidis, as is described in the Examples section which follows and Table 1 below.

Peptide 16 has at least 9 amino acid residues and less than 51 amino acid residues, and includes an amino acid sequence selected from SEQ ID NOs: 1-5. Preferably, peptide 16 includes the amino acid sequence set forth in SEQ ID NO: 1.

Preferably, the amino acid sequence of a peptide of the present invention which includes an amino acid sequence selected from SEQ ID NO: 1, 2, 3, 4 or 5 only includes the amino acid sequence set forth by SEQ ID NO: 1, 2, 3, 4 or 5, respectively. Preferably, a peptide of the present including an amino acid sequence set forth by SEQ ID NO: 1 is amidated. Preferably, an amidated peptide of the present including an amino acid sequence set forth by SEQ ID NO: 1 is not chemically modified at the N-terminus with a group including a carbon atom. Alternately, a peptide of the present invention including an amino acid sequence set forth by SEQ ID NO: 1 is chemically modified as described in Table 1.

Preferably the peptide of the present invention is chemically modified as described in Table 1 below. As is described in Example 1 of the Examples section which follows and Table 1 below, the peptide utilized by the present invention has potent antimicrobial activity against pathogens such as methicillin-resistant S. aureus and methicillin-resistant S. epidermidis, and as such, medical implants/devices of the present invention having surfaces coated with such peptides of the present invention can be used to optimally prevent infection with such pathogens following implantation of such medical implants/devices in-vivo.

TABLE 1
Peptides of the present invention, preferred chemical modifications
thereof, and relative anti-microbial capacities thereof.

| | |
|---|---|
| Preferred chemical | |
| modifications** | MIC*** |

| Peptide* | N-terminal | C-terminal | (μM) |
|---|---|---|---|
| ALWKTLLKICVLICA | H— | —CONH₂ | 9 ± 3 |
| (SEQ ID NO: 1) | C2— | —CONH₂ | 25 |
| C4— | —CONH₂ | 12 | |
| C6— | —CONH₂ | 2.2 ± 0.8 | |
| C8— | —CONH₂ | 1.5 | |
| C10— | —CONH₂ | 1.5 | |
| C12— | —CONH₂ | 4.5 ± 1.5 | |
| NC12— | —CONH₂ | 0.75 | |
| H— | —C12 | 6 | |
| H— | —C12N | 3 | |
| AKLVKKLLTKWLA | NC12— | —CONH₂ | 1.5 |
| (SEQ ID NO: 2) | | | |
| KALWKTLLKKVLKA | NC12— | —CONH₂ | 1.5 |
| (SEQ ID NO: 3) | | | |
| ALWKTLLKKV | C12 | —CONH₂ | 3 |
| (SEQ ID NO: 4) | | | |
| TLLKKVLKA | C12 | —CONH₂ | 1.5 ± 0.75 |
| (SEQ ID NO: 5) | | | |

*Peptide sequences having —CONH₂ are amidated. Peptide sequences starting with C2—, C4—, C6—, C8—, C10— or C12— are acylated at the amino end via amide bond, where the number following the C indicates the number of carbon atoms in the acyl group backbone (for example, C2—, acetyl—; C4—, butyry—; C12—, dodecanoyl and so on). Similarly, peptide sequences starting with
**Peptide modification legend:
—CONH2, carboxy terminal amidation;
C2—, C4—, C6—, C8—, C10—or C12—, amino terminal acylation via amide bond, where the number following the C indicates the number of carbon atoms in the acyl group backbone (for example, C2—, acetyl—; C4—, butyry—; C12—, dodecanoyl and so on);
NC12—, aminolauryl—/aminododecanoyl— group at the amino terminus;
—C12, dodecanoyl— group at the carboxy terminus;
—C12N, aminolauryl—/aminododecanoyl— group at the carboxy terminus;
SEQ ID NO: 1 has an amino acid sequence corresponding to that of amino acid residues 1-13 of dermaseptin S4, with a substitution at position 4 relative to the wild-type sequence;
SEQ ID NO: 2 has the reverse amino acid sequence of SEQ ID NO: 1;
SEQ ID NO: 3 has an amino acid sequence corresponding to SEQ ID NO: 2, with an additional Lys residue at the N-terminal;
SEQ ID NO: 4 has an amino acid sequence corresponding to that of amino acid residues 1—10 of dermaseptin S4 with a substitution at position 4 relative to the wild-type sequence;
SEQ ID NO: 5 has an amino acid sequence corresponding to that of amino acid residues 5—13 of dermaseptin S4;
***MIC, minimal inhibitory concentration assayed against S. aureus as previously described (Dagan et al., 2002. Antimicrobial Agents & Chemotherapy 46, 1059-66; Efron, L. et al., 2002. J. Biol. Chem. 277:24067-72).

Peptide 16 is preferably synthesized as described in the Examples section which follows. Alternately, ample guidance for synthesizing peptide 16 is available in art (refer, for example, to Kustanovich, I. et al., 2002. J. Biol. Chem. 277: 16941-51).

As is provided in Table 1 above, various configurations of peptide 16 have varying antimicrobial activities (MIC), and as such peptide 16 may be advantageously selected having a desired antimicrobial activity.

Surface 14 may include any combination of different peptides having an amino acid sequence selected from SEQ ID NOs: 1-5.

As used herein, the term “peptide” includes native peptides (such as polypeptide degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body, or more capable of being suitably cross-linked to surface 14. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Peptides of the present invention may be modified to include terminally groups such as an amine, an acyl, an aminoacyl, Fmoc, Boc and the like.

Methods for preparing peptidomimetic compounds are well known in the art and are specified, for example, in Quantitative Drug Design, C. A. Ramsden Gd., Chapter 17.2, F. Choplin Pergamon Press (1992).

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, include, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 2 and 3 below list naturally occurring amino acids (Table 2) and non-conventional or modified amino acids (Table 3) which can be used with the present invention.

TABLE 2
Naturally occurring amino acids.

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Tbr | T |
| Tryptophan | Trp | W |
| Tyrosine | Tyr | Y |
| Valine | Val | V |
| Any amino acid as | Xaa | X |
| above | | |

TABLE 3
Non-conventional or modified amino acids.

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cystene | Dcys | L-N-methylnorleucine | Nmnle |
| D-glutamine | Dgln | L-N-methylnorvaline | Nmnva |
| D-glutamic acid | Dglu | L-N-methylomithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanme | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-αnaphthylalnine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalamne | Anap |
| D-α-methyllysine | Dmlys | N-benzylglycine | Nphe |
| D-α-methylmethionine | Dmmet | N-(2-carbamylethyl)glycine | Ngln |
| D-α-methylamitbine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptopban | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Thug | N-(thioniethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamato | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methyiserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The peptides of the present invention can be utilized in a linear or cyclic form.

A peptide can be either synthesized in a cyclic form, or configured so as to assume a cyclic structure when attached and linear form when released. For example, a peptide according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2-)n-S—CH-2-C—, wherein n=1 or 2, which is possible, for example, through incorporation of Cys or homoCys and reaction of its free SH group with, e.g., bromoacetylated Lys, Om, Dab or Dap. Furthermore, cyclization can be obtained, for example, through amide bond formation, e.g., by incorporating Glu, Asp, Lys, Om, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH2)n-COOH)—C(R)H—COOH or H—N((CH2)n-COOH)—C(R)H—NH2, wherein n=14, and further wherein R is any natural or non-natural side chain of an amino acid.

For guidance regarding peptide chemistry, refer, for example to the extensive guidelines provided by The American Chemical Society (http://www.chemistry.org/portal/Chemistry). One of ordinary skill in the art, such as, for example, a chemist, will possess the required expertise for practicing chemical techniques suitable for obtaining peptides of the present invention.

Surface 14 may be contacted with peptide 16 so as to achieve coating of surface 14 with peptide 16 using any of various standard art methods. Surface 14 may be coated either non-covalently or covalently with peptide 16, depending on the desired binding characteristics. Preferably surface 14 is non-covalently coated with peptide 16. Preferably, surface 14 is coated with peptide 16 by contacting surface 14 with peptide 16. Contacting surface 14 with peptide 16 is preferably effected by exposing surface 14 to a solution of containing peptide 16 at a concentration selected from a range of 1 to 500 micrograms per milliliter, more preferably 5 to 400 micrograms per milliliter, more preferably 10 to 300 micrograms per milliliter, more preferably 20 to 200 micrograms per milliliter, and more preferably 30 to 100 micrograms per milliliter. Most preferably the concentration is about 50 micrograms milliliter.

Preferably, peptide 16 and the antibiotic is included in surface 14 in such a way as to enable slow release under relevant physiological conditions. For example, where the medical implant/device is a vascular graft, peptide 16 is included in surface 14 in such a way as to enable slow release under physiological conditions present in blood and/or in the tissues surrounding the graft, preferably both. Slow release of peptide 16 from surface 14 may be achieved using a surface which includes in its composition the polypeptide binding agents or synthetic binding agents described hereinbelow.

Peptide 16 may be coated onto surface 14 via a ligand attached to surface 14 which can bind the peptide, or an affinity tag attached to the peptide. Such a configuration enables rapid coating of surface 14 with peptide 16, and as a consequence, extends the shelf life of the coated surface. Numerous affinity-tag/ligand systems are available for practicing such a coating technique.

Examples of affinity tags include streptavidin tags, polyhistidine tags (His-tags), streptavidin tags (Strep-tags), biotin tags, epitope tags, maltose-binding protein (MBP) tags, and chitin-binding domain (CBD) tags.

A His-tag is a peptide typically consisting of about six contiguous histidine amino acid residues having the capacity to specifically bind nickel-containing substrates. Ample guidance regarding the use of His-tags is available in the literature of the art (for example, refer to Sheibani N. 1999. Prep Biochem Biotechnol. 29, 77). An alternate suitable capture ligand for His-tags is the anti His-tag single-chain antibody 3D5 (Kaufmann, M. et al., 2002. J Mol Biol. 318, 135-47).

Examples of epitope tags include an 11-mer Herpes simplex virus glycoprotein D peptide, and an 11-mer N-terminal bacteriophage t7 peptide, being commercially known as HSVTag and t7Tag, respectively (Novagen, Madison, Wis., USA), and 10- or 9-amino acid c-myc or Hemophilus influenza hemagglutinin (HA) peptides, which are recognized by the variable regions of monoclonal antibodies 9E10 and 12Ca5, respectively.

A Strep-tag is a peptide having the capacity to specifically bind streptavidin. Ample guidance regarding the use of Strep-tags is provided in the literature of the art (see, for example: Schmidt, T G M. and Skerra, A. 1993. Protein Eng. 6, 109; Schmidt T G M. et al., 1996. Journal of Molecular Biology 255, 753-766; Skerra A. and Schmidt T G M., 1999. Biomolecular Engineering 16, 79-86; Sano T. and Cantor C R. 2000. Methods Enzymol. 326, 305-11; and Sano T. et al., 1998. Journal of Chromatography B 715, 85-91).

One of ordinary skill in the art, such as a chemist will possess the necessary expertise for conjugating the affinity tag to peptide 16 and for including the ligand in surface 14

Exposing surface 14 to the solution is preferably effected for a duration selected from a range of 0.05 to 50 hours, more preferably 0.5 to 40 hours, more preferably 1 to 30 hours, more preferably 2 to 20 hours, and more preferably 3 to 10 hours. Most preferably, the duration is about 5 hours.

As used herein the term “about” refers to ±10 %.

Exposing surface 14 to the solution is preferably effected as described in Example 1 of the Examples section which follows, so as to achieve suitable non-covalent coating of surface 14 with peptide 16.

Preferably, the solution further comprises the antibiotic so to enable coating of surface 14 therewith. The concentration of the antibiotic in the solution is preferably selected from a range of 0.5 to 50 micrograms per milliliter, more preferably 1 to 40 micrograms per milliliter, more preferably 2 to 30 micrograms per milliliter, more preferably 3 to 20 micrograms per milliliter, and more preferably 4 to 10 micrograms per milliliter. Most preferably, the concentration of the antibiotic in the solution is about 5 micrograms per milliliter.

As is described in Example 1 of the Examples section below, a medical implant/device of the present invention having surface 14 which is coated with peptide 16 and the rifampin is optimally resistant to in-vivo infection with microbial pathogens, such as methicillin-resistant staphylococci.

Preferably, surface 14 is coated with peptide 16 at a surface density selected from a range of 0.4 to 275 micrograms per square centimeter, more preferably 4 to 27.5 micrograms per square centimeter.

Various techniques may be employed for contacting surface 14 with peptide 16 so as to coat surface 14 therewith. For example, surface 14 may be non-covalently coated with peptide 16 via vapor phase deposition. Currently available vapor phase deposition systems include Specialty Coating Systems™ (100 Deposition Drive, Clear Lake, Wis. 54005), Para Tech Coating™, Inc. (35 Argonaut, Aliso Viejo, Calif. 92656) and Advanced Surface Technology™, Inc. (9 Linnel Circle, Billerica, Mass. 01821-3902). Alternately, suitable coating techniques include spraying, and the like (see U.S. Pat. No. 5,873,904 for further detail).

Coating of surface 14 may with peptide 16 may be achieved via any of techniques commonly practiced in the art.

Covalent immobilization of peptide 16 or the antibiotic to Dacron may be performed using any of various standard art methods (refer, for example, to Ito R K. et al., 1991 2(1):77-81; Holt D B. et al., 1994. ASAIO J. 40:M858-63).

Surface 14 may be coated with peptide 16 or the antibiotic as described in Okahara et al., Eur. J. Vasc. Endovasc. Surg. 9: 408 (1995).

Coating of surface 14 with peptide 16 can be effected by any direct or indirect conjugation method, which is selected primarily according to the nature of the substrate to be coated.

Generally, metal particles can bind organic moieties through either non-covalent (i.e., electrostatic) or covalent interaction. Non-covalent binding is preferably used when low binding of an organic moiety per metal molecule is desired.

U.S. Pat. No. 5,728,590, describes covalent binding methods of organic moieties to metallic clusters or colloids which can be used with the present invention. The process involves synthesis of the metal colloid (For example, HauC14 (0.01%) in 0.05M sodium hydrogen maleate buffer (pH 6.0), with 0.004% tannic acid.) in the presence of a suitable polymer. The polymer may be chosen from a linear or branched group with functional groups attached, such as polyamino acids, polyethylene derivatives, other polymers, or mixtures thereof. A second method is to synthesize the metal particle first, e.g., by combining 0.01% HauC14 with 1% sodium citrate with heating. Once gold colloid of the desired size is formed, it is coated with a polymer by mixing the two together and optionally warming to 60-100° C. for several minutes. The polymer coating may be further stabilized by (i) microwave heating, (ii) further chemical crosslinking, e.g., by glutaraldehyde or other linkers, or by continued polymerization adding substrate molecules for a brief period. N,N′-methylene bis acrylamide, can be used to covalently stabilize the polymer coating. Photocrosslinking may also be used.

Once formed, the functionalized polymer coating can be used to attach peptide 16 and/or the antibiotic.

It will be appreciated that the synthesis method described hereinabove is advantageous, since coupling may be done mildly, in physiological buffers if desired, using standard crosslinking technology.

Conjugation of molecules such as peptide 16 and/or the antibiotic to a synthetic carbon polymer surface can be effected via any approach well known in the art. U.S. Pat. No. 6,338,904 provides a comprehensive description of suitable approaches.

The following section provides detail of several approaches, which can be used by the present invention to conjugate peptide 16 and/or the antibiotic to a synthetic carbon polymer surface.

(i) Binding through a chemical linking moiety. The chemical linking moiety has a structure represented by: A-X—B, wherein A is a photochemically reactive group, B is a reactive group which responds to a different stimulus than A and X is a non-interfering skeletal moiety, such as a C1-C10 alkyl. Covalent binding of peptide 16 and/or the antibiotic to the surface of the medical device is effected via the linking moiety.

(ii) Covalent binding to an amine-rich material, (e.g., a polyurethaneurea) modified with hydrophobic groups (U.S. Pat. No. 4,720,512).

(iii) Ionic binding via a quaternary ammonium compound. See U.S. Pat. Nos. 4,229,838, 4,613,517, 4,678, 660, 4,713,402, and 5,451,424 for details.

(iv) covalent binding through a hydrophilic spacer reacted with one or more of a reactive functional group overhanging from a polymer backbone (U.S. Pat. No. 6,338,904).

Peptide 16 or the antibiotic may be included in/co-fabricated with surface 14 via any of numerous strategies known in the art.

Polypeptide binding agents may be employed in order to create localized concentrations of peptide 16 or the antibiotic in surface 14. These agents, may be either protein or synthetic-based, are embedded within the biomaterial matrix thereby either “trapping” or ionically binding the antibiotic. The basement membrane protein collagen may be used to include peptide 16, as previously described for rifampin [Krajicek et al., J. Cardiovasc. Surg. 10: 453 (1969); Goeau-Brissonniere, O., J. Mal. Vasc. 21: 146 (1996); Strachan et al., Eur. J. Vasc. Surg. 5: 627 (1991)]. Fibrin, either as a pre-formed glue or in pre-clotted blood, may be utilized as a binding agent, as previously described for various antibiotics [Haverich et al., J. Vasc. Surg. 14: 187 (1992); McDougal et al., J. Vasc. Surg. 4: 5 (1986); Powell et al., Surgery 94: 765 (1983); Greco et al., J. Biomed. Mater. Res. 25: 39 (1991)]. Albumin or gelatin may be used to include peptide 16 or the antibiotic in surface 14, as previously taught for rifampin and vancomycin [Muhl et al., Ann. Vasc. Surg. 10: 244 (1996); Sandelic et al., Cardiovasc. Surg. 4: 389 (1990)]. Any of various synthetic binding agents may be employed to include peptide 16 or the antibiotic in surface 14 [refer, for example to Shenk et al., J. Surg. Res. 47: 487 (1989); Suzuki et al., ASAIO J. 43: M854 (1997)]. Alternately, peptide 16 or the the antibiotic may be cofabricated with device body 12 [refer, for example to Golomb et al., J. Biomed. Mater. Res. 25: 937 (1991); Whalen et al., ASAIO J. 43: M842 (1997)].

The medical implant/device of the present invention may be designed and configured as essentially any desired type of medical implant/device, examples of which are listed below.

The medical implant/device may be configured as a prosthesis such as a vascular graft, a bypass conduit, a vascular sidewall patch, a vascular support bandage, a catheter, a catheter wall/lining, a catheter sheeting/film, a wire guide, a cannula, a stent, a cardiac pacemaker lead or lead trap, a cardiac defibrillator lead or lead tip, a heart valve or an orthopedic device, appliance, implant or replacement or a hemodialyzer.

The medical implant/device may be configured as a mechanical device such as a heart valve, a cardiac valve sewing ring, a blood flow check valve, a ventricular assist device, a whole artificial heart, or a respirator tube.

The medical implant/device may be configured as a fiber, such as a wound treatment dressing/film/sheet, a gauze pad, a surgical sponge, or a suture material.

The medical implant/device may be a dental implant, a subcutaneous cosmetic implant or a contact lens.

The medical implant/device can be configured as any combination or a portion of the above described medical implants/devices.

The present invention therefore provides a method of preventing microbial infection in a subject in need of implantation of a medical implant/device. The method is effected by administering to the subject a suitably configured medical implant/device of the present invention.

It will be appreciated that by virtue of the optimal antimicrobial properties of the medical implants/devices of the present invention, the present invention enables treatment of a subject by administration of a medical implant/device with optimally low risk of microbial infection associated with such implantation.

One of ordinary skill in the art, such as a physician, in particular a physician specialized in the tissue region of a subject in which a particular type of medical implant/device is to be implanted, will possess the expertise required for suitably administering such a medical implant/device to the subject.

Preferably, the method is used to administer a vascular graft to a subject in need of implantation thereof. A surgeon, such as a cardiac or vascular surgeon, will possess the necessary expertise for suitable administration of a vascular graft of the present invention to a subject having a medical condition requiring implantation of a vascular graft.

Examples of medical conditions amenable to treatment via administration of a vascular graft include vascular ischemia, thromboembolism, myocardial infarction, atherosclerosis, arterial aneurysm, vascular hemorrhage, vascular injury, and the like.

Thus, as is described and illustrated in Example 1 of the Examples section which follows, the present invention teaches the fabrication of, and provides, a medical implant/device which can be used to practice in-vivo implantation of medical implants/devices such that the implantation is associated with optimally low risk of infection by microbial pathogens. In particular, the present invention provides vascular grafts whose implantation is associated with optimally low risk of infection with methicillin-resistant strains of S. aureus or S. epidermidis relative to the prior art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al, “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Optimal In-Vivo Prevention of Infection of Medical Implants by Antibiotic-Resistant Bacterial Pathogens Using Dermaseptin Derivative and RIP Peptides

Background: Infection by dangerous microbial pathogens are currently responsible for numerous highly debilitating and/or lethal complications, which are difficult or impossible to treat, following administration of medical implants/devices. In particular, infection of synthetic carbon polymer grafts, such as vascular grafts composed of Dacron, by staphylococci, such as methicillin-resistant S. aureus or S. epidermidis, remains a devastating potential complication following implantation of such grafts. A potentially potent strategy which has been proposed for preventing such infections involves treating such grafts with antimicrobial peptides. While a variety of such approaches have been attempted, none have so far succeeded in enabling fabrication of medical implants/devices such as Dacron vascular grafts presenting optimally low risk of infection post-implantation by methicillin-resistant S. aureus or S. epidermidis. While reducing the present invention to practice as described below a method of producing such grafts was unexpectedly uncovered, thereby overcoming the limitations of the prior art.

Materials and Methods:

Peptide synthesis: The 13-residue dermaseptin S4-derived peptide K₄-S4(1-13) having the amino acid sequence ALWKTLLKKVLKA (SEQ ID NO: 1), and the 7-residue RNAIII-inhibiting peptide (abbreviated RIP) having the amino acid sequence YSPWTNF (SEQ ID NO: 6) were respectively synthesized as amidated peptides (amidated peptide K₄-S4(1-13) is designated in the art as K₄-S4(1-13)ₐ, and is abbreviated herein as DD13) by the solid phase method, as previously described (Kustanovich, I. et al., 2002. J. Biol. Chem. 277: 16941-51), via 9-fluorenylmethyloxycarbonyl (Fmoc) active ester chemistry using a fully automated programmable Model 433A Peptide Synthesizer (Applied Biosystems). Peptide DD13 corresponds to amino acid residues 1-13 of dermaseptin S4, with a substitution to a Lys residue at position 4. Among the dermaseptin S4 derivatives tested in published studies, DD13 was found to be the smallest derivative that combines low toxicity and efficient broad-spectrum antimicrobial activity in culture (Feder, R. et al., 2001. Peptides. 22: 1683-90). To obtain the amidated peptides, 4-methylbenzhydrylamine (MBHA)-resin (Novabiochem, Germany) was used. The crude peptides were extracted from the resin with 30 percent acetonitrile in water and purified to chromatographic homogeneity in the range of 98 to greater than 99 percent by reverse-phase HPLC (Alliance-Waters). HPLC runs were performed on a semipreparative C4 column (Vydac) using a linear gradient of acetonitrile in water (1 percent/minute), both solvents containing 0.1 percent trifluoroacetic acid. The purified peptides were subjected to amino acid analysis and electrospray mass spectrometry in order to confirm their composition. Peptides were stored as a lyophilized powder at −20 degrees centigrade. The amidated forms of the peptides were employed in the experiments described herein.

Bacterial strains: Methicillin-resistant S. aureus (abbreviated MRSE) ATCC 43300 was commercially purchased from Oxoid S.p.A., Milan, Italy. Methicillin-resistant S. epidermidis (abbreviated MRSE) is a clinical strain from the Institute of Infectious Diseases and Public Health, University of Ancona, Italy.

Animals: Adult male Wistar rats, 250-300 gr (I.N.R.C.A. I.R.R.C.S. animal facility, Ancona) were used, with 15 animals per experimental group.

Bacterial strain antibiotic suscepdbilty analysis: The antimicrobial susceptibilities of the bacterial strains were determined by using the microbroth dilution method, according to the procedures outlined by the National Committee for Clinical Laboratory Standards (U.S.A.). The minimal inhibitory concentration (MIC) was taken as the lowest antibiotic concentration at which observable growth was inhibited. Experiments were performed in triplicate.

Mammalian graft infection model: Rats were anesthetized and their hair of the back was shaved and the skin cleansed with 10 percent povidone-iodine solution. One subcutaneous pocket was made on each side of the median line by a 1.5 cm incision. Aseptically, 1 square centimeter sterile albumin-sealed Dacron™ grafts (Albograft™, Sorin Biomedica Cardio, S.p.A., Saluggia VC, Italy) were implanted into the pockets. Prior to implantation grafts were soaked for 20 minutes in sterile antibiotic solutions. The pockets were closed by means of skin clips and saline solution (1 mL) containing methicillin-resistant S. aureus (MRSA) or S. epidermidis (MRSE) at a concentration of 2×10⁷ colony-forming units (CFU)/mL was inoculated onto the graft surface by using a tuberculin syringe to create a subcutaneous fluid-filled pocket. The animals were returned to individual cages and thoroughly examined daily. All grafts were explanted at 7 days following implantation.

Assessment of the infection: The explanted grafts were placed in sterile tubes, washed in sterile saline solution, placed in tubes containing 10 mL of phosphate-buffered saline solution and sonicated for 5 minutes to remove the adherent bacteria from the grafts (Balaban, N. et al, 2003. Kidney Int. 63: 340-345). Quantitation of viable staphylococci was performed by culturing serial dilutions (0.1 mL) of the bacterial suspensions on blood agar plates at 37 degrees centigrade for 48 hours. The organisms were quantitated by counting the number of colony-forming units (CFUs) per plate. The limit of detection for this method was approximately 10 CFU/mL.

Peptide: Dacron™ binding assay: To quantitate the capacity of the peptides to bind to synthetic grafts, 1 square centimeter sheets of collagen-sealed Dacron™ graft (Albograft™, Sorin Biomedica Cardio, S.p.A., Saluggi VC, Italy) were soaked for 0.5 or 5 hours in at room temperature in 1 mL of saline solution containing 50 micrograms peptide/mL in quadruplicate. Following the incubation period the Dacron™ grafts were removed and residual peptide (unbound fraction) was quantitated via reversed phase-HPLC analysis, as described above. Peptide identification was based on retention time and spectral analysis. The amount of unbound peptide was calculated by area integration of the UV absorbing peak (OD 220 nm) and comparison with standard curves of known concentrations for each peptide (Feder, R. et al., 2000. J. Biol. Chem. 275: 4230-38).

RNAIII synthesis and bacterial growth: Cultures of S. aureus (20 million cells in a volume of 30 microliters) containing an expression construct for expression of the fusion protein maiii::blaZ (described in Gov, Y. et al., 2001. Peptides. 22: 1609-20 were grown for 2.5 hours with 5 microliters peptide solution or control buffer. A 5 microliter sample was taken from the cultures, diluted in saline, and streaked on LB agar plates to quantitate CFUs. The remainder of the cultures were used to determine RNAIII synthesis (beta-lactamase activity). This was performed by adding a substrate of beta-lactamase (nitrocefin), and determining OD 490 nm/OD 650 nm, as previously described (Gov, Y. et al., 2001. Peptides. 22: 1609-20).

Statistical analysis: MIC values are presented as the geometric mean of three separate experiments. Quantitative culture results from all groups are presented as mean +standard deviation and the statistical comparisons between groups were made using analysis of variance (ANOVA) on the log-transformed data. Significance was accepted when the P value was less than/equal to 0.05.

Experimental Results:

Peptide DD13 optionally prevents infection of grafts in-vivo with antibiotic-resistant Staphylococci strains: Peptides DD13 and RIP were tested for their capacity to prevent staphylococcal graft-associated infections in-vivo. Collagen-coated Dacron™ grafts were soaked in solution containing 10, 20 or 50 micrograms/mL of either RIP or DD13 and were implanted in subcutaneous pockets in rats. Staphylococcal strains MRSA or MRSE were injected into the pockets, the implants were removed after a week, and their bacterial load was determined. The study included a negative control group (untreated graft with no bacterial challenge) and a positive control group (untreated graft with bacterial challenge). None of the animals included in the negative control group had anatomic or microbiological evidence of graft infection (no graft contamination). All rats included in the positive control groups that were implanted with untreated grafts and challenged with MRSA or MRSE (total of 30 rats) demonstrated evidence of graft infection, with quantitative culture results showing 4.4×10⁶±1.2×10⁶ CFU/ml and 6.9×10⁶±1.8×10⁶ CFU/ml graft, respectively.

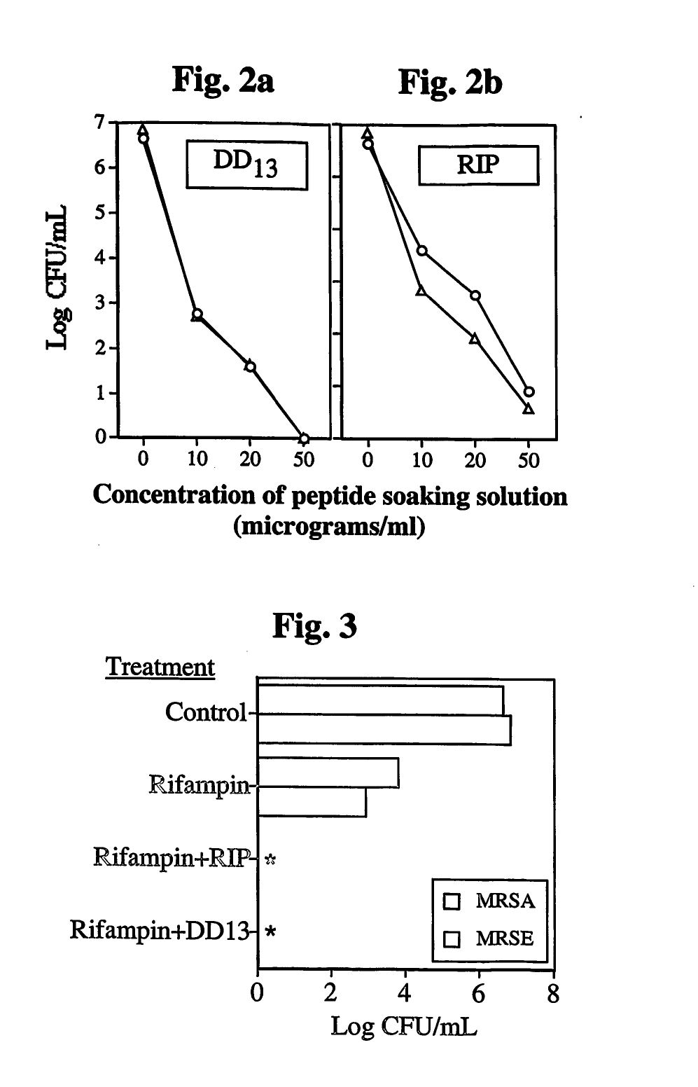

As is shown in FIGS. 2a, grafts presoaked in DD13 solution and challenged with MRSE or MRSA demonstrated optimally reduced levels of infection in a very strong antibiotic dose-dependent fashion as compared to grafts presoaked in RIP (FIG. 2b). Specifically, as is shown in FIG. 1a, grafts presoaked in 10, 20 or 50 micrograms/mL DD13 solution and challenged with MRSA displayed optimally low to insignificant levels of infection with 5.2×10²±1.6×10², 4.0×10¹±1.7×10¹ CFU/ml and negative quantitative cultures, respectively. Similarly, grafts presoaked in 10, 20 or 50 micrograms/mL DD13 solution and challenged with MRSE, demonstrated reduced or no evidence of infection with 5.2×10²±1.6×10², 4.4×10¹±1.3×10¹ CFU/ml and negative quantitative cultures, respectively. As is shown in FIG. 2b, grafts presoaked in 10, 20 or 50 micrograms/mL RIP solution and challenged with MRSE displayed 6.9×10³±1.9×10³, 8.5×10²±2.0×10² and 3.9×10¹±1.6×10¹ CFU/ml, respectively, and those challenged with MRSA demonstrated displayed 4.1×10⁴±7.1×10³ CFU/ml, 5.9×10³±1.7×10³ and 8.4×10¹±3.6×10¹ CFU/ml respectively.

It should be noted is that all agents used did not show any signs of toxicity and none of the animals included in any group died or had clinical evidence of drug related adverse effects, such as local signs of perigraft inflammation, anorexia, vomiting, diarrhea, and behavioral alterations. Reduction in bacterial load was significant (p<0.05) in all experimental groups when compared to positive control groups.

Treatment with peptide DD13 in combination with rifampin completely prevents/treats infection of synthetic grafts implanted in-vivo with antibiotic-resistant Staphylococcus strains: The effectiveness of treatment with DD13 or RIP of Dacron™ grafts implanted in rats and challenged with MRSA and MRSE was compared with that of the conventional antibiotic, rifampin (Sardelic, F. et al., 1996. Cardiovasc. Surg. 4: 389-392). Experimental groups received grafts pre-soaked either with 5 micrograms/mL rifampin alone, or with rifampin in combination with DD13 or RIP at 10 micrograms/mL. As can be seen in FIG. 3, the group of rifampin-treated rats showed only about 50 percent reduction in numbers of PFUs, compared to control, with 6.7×10³±9.1×10² CFU/mL following challenge with MRSA and 8.8×10²±3.0×10² CFU/mL following challenge with MRSE. However, in dramatic contrast, graft treatment with rifampin in combination with DD13 yielded negative quantitative cultures and no evidence of infection, demonstrating for the first time the optimal effectiveness of the combination of DD13 and rifampin in preventing infection of in-vivo implants.

It should be noted is that all agents used did not show any signs of toxicity and none of the animals included in any group died or had clinical evidence of drug related adverse effects, such as local signs of perigraft inflammation, anorexia, vomiting, diarrhea, and behavioral alterations. Reduction in bacterial load was significant (p<0.05) in all experimental groups when compared to positive control groups.

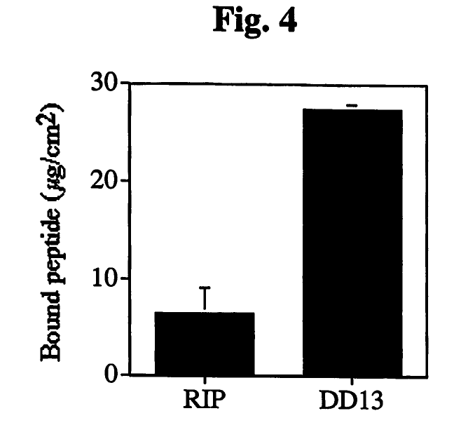

Peptide DD13 has 3-fold higher Dacron-binding capacity than RIP: In order to correlate the observed in-vivo activity of peptide DD13 or RIP and, and the amounts of these peptides present on the Dacron™ grafts, grafts were soaked in 50 micrograms/mL peptide solution, as described above, the grafts were removed from the solutions, and the solutions were subjected to HPLC analysis to quantitate the amount of Dacron™-bound peptide by deduction from the calculated amount of residual unbound fraction. The results are shown in FIG. 4. RIP was observed to bind with a mean bound amount of 6.5±2.5 micrograms peptide per square centimeter, and DD13 was found to bind at about 3-fold higher levels than RIP with a mean bound amount of 27±0.5 microgram/square centimeter. The data indicates that longer soaking time periods enable uptake of larger amounts of each peptide, with, for instance, nearly 100 percent of 50 micrograms of DD13 being found to bind after 5 hours soaking (data not shown). While these experiments indicate that longer soaking time may be employed to achieve higher levels of peptide binding to the grafts, they also demonstrate that the higher protective efficacy of DD13 as compared to RIP is not due to its relative concentration but rather to its specific activity, as discussed further below.

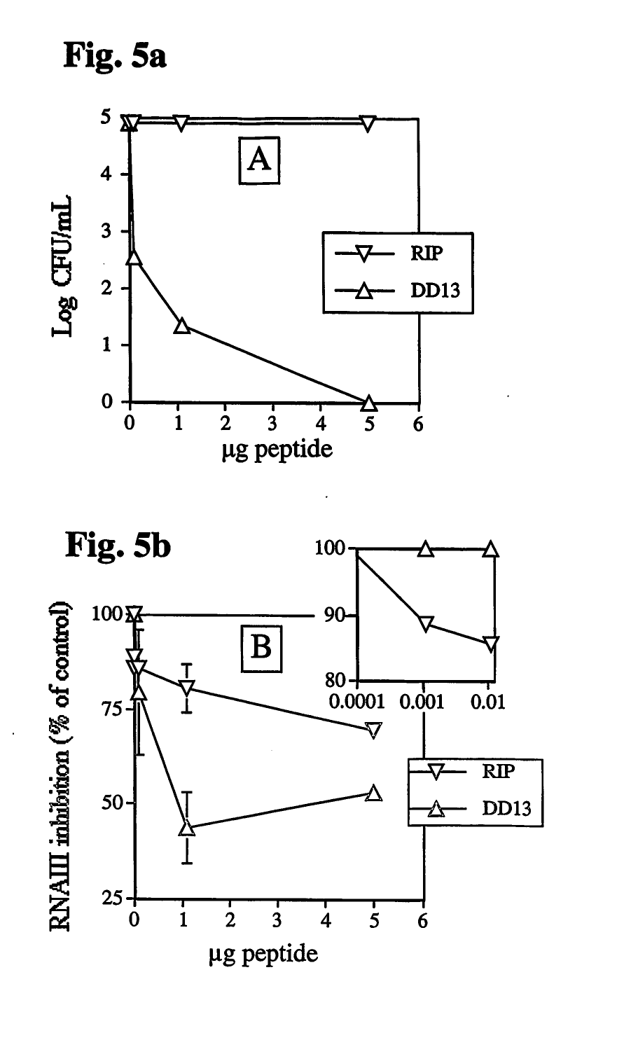

Peptide DD13 exhibits optimal in-vitro bactericidal activity against methicillin-resistant S. aureus relative to peptide RIP: In order to analyze the molecular mechanisms involved in their observed antimicrobial activity, DD13 was investigated for its effect in-vitro on RNAIII synthesis, a phenomenon known to be inhibited by RIP, and for bacterial proliferation, a phenomenon known to be inhibited by peptide DD13. These experiments were performed by growing MRSA or MRSE cells containing an maiii::blaZ fusion construct in the presence of various concentrations of RIP or DD13 peptide. Cultures were monitored for RNAIII synthesis by a colorimetric method using nitrocefin as a substrate while the peptide's effect on bacterial viability was assessed by performing CFU counts using conventional microbiological methods. DD13 was found to display potent bactericidal activity whereas RIP was virtually inactive (FIG. 5a). According to the broth-micro dilution method, DD13 exhibited a MIC at 2 micrograms/mL (1.3 micromolar) for both staphylococcal strains (as compared to susceptibility to rifampin of MIC values of 0.5 microgram/mL for both of the organisms). RIP did not demonstrate any In-vitro bactericidal activity against either of the two strains, when tested at concentrations up to 128 micrograms/mL (data not shown). As shown in FIG. 5b, RIP efficiently inhibited RNAIII synthesis while DD13 appeared to be more efficient than RIP. However, closer inspection of the data revealed that at high peptide concentrations, most of the inhibitory activity observed was attributable to cell death. Moreover, at low peptide concentrations, where no cell death occurred, DD13 was unable to affect RNAIII synthesis (inset).

Discussion: In order to demonstrate efficacy in preventing bacterial adhesion and biofilm formation in-vivo, a well-characterized experimental Dacron™ graft rat model was used. For comparison purposes the antibiotic rifampin was chosen for its current utilization in clinical practice against Staphylococci (Sardelic, F. et al., 1996. Cardiovasc. Surg. 4: 389-392). Rifampin was found to be effective in the presently described experimental model, as expected from the literature (Balaban, N. et al., 2003. J Infect Dis. 187: 625-30; Giacometti, A. et al., 2003. Antimicrobial Agents and Chemotherapy, in Press). As with peptide RIP or DD13, rifampin used alone did not eradicate bacterial infection. Surprisingly complete and optimal eradication of bacterial infection was synergistically achieved when DD13 was combined with rifampin, with antiinfective activity being superior to that obtained when RIP was combined with rifampin. The reason that RIP was less effective than DD13 when used alone could be due to the actual amount of peptide bound to the graft. It is estimated that soaking the graft in 50 micrograms/mL RIP resulted in only 6.5±2.5 micrograms RIP bound to the graft prior to implantation, which may not be enough to prevent adhesion of the 10 million cells that were injected. The present inventors therefore predict that coating of grafts with increasing concentrations of DD13, as may be obtained using increased soaking time, may be used achieve increased anti-bacterial effect. In any case, it should be noted, however, that levels of bacterial inoculation of grafts as high as those employed in the presently described experiments is highly unlikely to occur via simple contamination in the clinical setting. Hence, the present inventors predict that, in the clinical setting, medical implants coated with the amounts of bound peptides utilized in the present in-vivo experiments will be highly efficient in preventing infection with bacterial pathogens, such as S. aureus. While DD13 had about threefold higher binding capacity to Dacron™ than RIP, efficacy in-vivo was at least tenfold higher at each one of the concentrations used, indicating that the high efficacy of DD13 in-vivo is not due to its relative concentration on the graft but rather to its specific activity. The use of DD13 is therefore preferable over that of RIP due to the former's ability to kill bacteria and simultaneously neutralize the threat that the bacteria might release endotoxins which can cause septic shock upon their introduction into the bloodstream. Antimicrobial peptides such as dermaseptin, of which DD13 is an optimally active derivative, are known for their ability to bind endotoxins and neutralize them and thereby prevent septic shock.

Conclusion: The presently disclosed experimental results teach for the first time that coating a medical implant/device, such as a Dacron graft having a collagen-coated surface, with dermaseptin S4-derived peptides, such as amidated DD13, alone or optimally in combination with rifampin, can be used to optimally reduce the risk that in-vivo implantation of such an implant/device will be associated with infection by dangerous microbial pathogens, such as methicillin-resistant S. aureus or S. epidermidis strains. As such, the presently described peptide-coated medical implants/devices can be used to optimally treat/prevent infections of synthetic grafts in the clinical setting, such as infection of vascular Dacron™ grafts with methicillin-resistant S. aureus or S. epidermidis following implantation of such grafts in human patients in the clinical setting relative to the prior art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by its accession number was specifically and individually indicated to be incorporated herein by reference. In addition, citation or identification of any reference in this application shall not be construed as an admission that such reference is available as prior art to the present invention.

What is claimed is:

1. A medical device or implant comprising a body having at least one surface coated with, or including a peptide having at least 9 amino acid residues and less than 51 amino acid residues, said peptide including an amino acid sequence selected from the group consisting of SEQ ID NOs: 1-5.

2. The medical device or implant of claim 1, wherein said peptide is amidated.

3. The medical device or implant of claim 1, wherein said at least one surface is coated with said peptide at a surface density selected from a range of 0.4 to 275 micrograms per square centimeter.

4. The medical device or implant of claim 1, wherein said at least one surface is composed of a synthetic carbon polymer and/or a polypeptide.

5. The medical device or implant of claim 1, wherein the medical device or implant is a vascular graft.

6. The medical device or implant of claim 1, wherein said at least one surface is also coated with or also includes an antibiotic.

7. The medical device or implant of claim 6, wherein said antibiotic is rifampin.

8. A method of fabricating a medical device or implant capable of killing, or preventing a growth of, a microbial pathogen, the method comprising contacting at least one surface of a body of the medical device or implant with a peptide having at least 9 amino acid residues and less than 51 amino acid residues, said peptide including an amino acid sequence selected from the group consisting of SEQ ID NOs: 1-5, thereby rendering the surface of the medical implant capable of killing, or preventing the growth of, the microbial pathogen.

9. The method of claim 8, wherein said contacting said at least one surface of the medical device or implant with said peptide is effected by exposing said at least one surface of the medical device or implant with a solution of said peptide, wherein the concentration of said peptide in said solution is selected from a range of 1 to 500 micrograms per milliliter.

10. The method of claim 9, wherein said exposing said at least one surface of the medical device or implant with said solution of said peptide is effected for a duration selected from a range of 0.05 to 50 hours.

11. The method of claim 9, wherein said solution further comprises an antibiotic.

12. The method of claim 11, wherein said antibiotic is rifampin.

13. The method of claim 11, wherein a concentration of said antibiotic in said solution is selected from a range of 0.5 to 50 micrograms per milliliter.

14. The method of claim 8, wherein said at least one surface is composed of a synthetic carbon polymer and/or a polypeptide.

15. The method of claim 8, wherein the medical device or implant is a vascular graft.

16. The method of claim 8, wherein said peptide is amidated.

17. A method of preventing microbial infection in a subject in need of implantation of a medical implant, the method comprising administering to the subject a medical implant comprising a body having at least one surface, said at least one surface being coated with, or including a peptide having at least 9 amino acid residues and less than 51 amino acid residues, said peptide including an amino acid sequence selected from the group consisting of SEQ ID NOs: 1-5, thereby treating the subject in need thereof with the medical implant.

18. The method of claim 17, wherein said at least one surface is coated with said peptide at a surface density selected from a range of 0.4 to 275 micrograms per square centimeter.

19. The method of claim 17, wherein said at least one surface is composed of a synthetic carbon polymer and/or a polypeptide.

20. The method of claim 17, wherein said peptide is amidated.

21. The method of claim 17, wherein the medical implant is a vascular graft.

22. The method of claim 17, wherein said at least one surface is also coated with or also includes an antibiotic.

23. The method of claim 22, wherein said antibiotic is rifampin.


SEQUENCE LISTING (6 sequences)

SEQ ID NO: 1 - protein, 13 aa, Artificial sequence
  Synthetic peptide
ALWKTLLKKVLKA

SEQ ID NO: 2 - protein, 13 aa, Artificial sequence
  Synthetic peptide
AKLVKKLLTKWLA

SEQ ID NO: 3 - protein, 14 aa, Artificial sequence
  Synthetic peptide
KALWKTLLKKVLKA

SEQ ID NO: 4 - protein, 10 aa, Artificial sequence
  Synthetic peptide
ALWKTLLKKV

SEQ ID NO: 5 - protein, 9 aa, Artificial sequence
  Synthetic peptide
TLLKKVLKA

SEQ ID NO: 6 - protein, 7 aa, Artificial sequence
  Synthetic peptide
YSPWTNF